(19) United States
(12) Patent Application Publication
(10) Pub. No.: US 2025/0115626 A1
(43) Pub. Date: Apr. 10, 2025

(54) BIFUNCTIONAL LINKER COMPRISING METAALLYLSILANE AND METHOD FOR PREPARING SAME

(71) Applicant: INDUSTRY-UNIVERSITY COOPERATION FOUNDATION HANYANG UNIVERSITY ERICA CAMPUS, Gyeonggi-do (KR)

(72) Inventors: Sun Joon MIN, Seoul (KR); Won Cheol YOO, Gyeonggi-do (KR); Young Bok LEE, Seoul (KR); Kwang Suk JANG, Gyeonggi-do (KR); Jae Woon LEE, Gyeonggi-do (KR); Jeung Hwan KIM, Seoul (KR); Ji Won KIM, Gyeonggi-do (KR); Incheol HEO, Gyeonggi-do (KR)

(21) Appl. No.: 18/724,777

(22) PCT Filed: Nov. 21, 2022
(86) PCT No.: PCT/KR2022/018414

§ 371 Date: Jun. 27, 2024

(30) Foreign Application Priority Data
Dec. 29, 2021 (KR) 10-2021-0191441
Oct. 27, 2022 (KR) 10-2022-0140095

(51) Int. Cl. C07F 7/08; C01B 33/12

(52) U.S. Cl. CPC C07F 7/0812; C01B 33/12

(57) ABSTRACT

The present invention relates to a metaallylsilane-based linker compound, a method for preparing same, and the like. The present invention makes it possible to prepare a nanoprobe in which two or more functional molecules are incorporated in a silica nanoparticle that is modified with the linker compound by means of a one-pot process through a continuous bioorthogonal reaction. The nanoprobe of the present invention can be incorporated in a targeting ligand capable of delivering a drug to a specific location, a fluorescent dye for molecular imaging, a radioactive isotope, a magnetic substance, a drug for treatment, and the like, and thus can be used for in vivo molecular imaging, theranostics, and the like.

### TECHNICAL FIELD

The present disclosure relates to a bifunctional linker including methallylsilane, and a method for preparing the same, and the like.

### BACKGROUND ART

In a society that is changing into an aging society, the importance of disease prevention and early medical checkups is emerging, and thus the development of an early disease diagnosis system is becoming more important. Moving on from diagnostic systems using serum and body fluids, the development of biological diagnostic systems to determine the presence or absence of diseases within tissues and organs by applying them directly to the body is actively underway. In particular, efforts are being made to develop nano-sized inorganic and organic materials as biological diagnostic materials based on their unique structural properties, and among them, fluorescent nanomaterials are gaining popularity due to their high sensitivity and convenience.

Materials for in vivo molecular imaging must have excellent biocompatibility and be capable of targeting specific tissues and diseases. Having started recently, in order to improve the functionality of optical in vivo molecular imaging materials, the development of 1) fluorescence intensity amplification technology of imaging materials, 2) surface treatment technology and discovery of biocompatible materials for development of biocompatible nanomaterials, and 3) imaging material presenting tissue- and disease-specific targeting motifs is actively underway.

Among them, silica nanoparticles not only have high stability and biocompatibility, but also allow the introduction of large amounts of fluorescent substances to produce a strong fluorescence signal. In addition, they may prevent rapid photobleaching by oxygen, and thus have high photostability. Furthermore, it is easy to introduce various functional molecules into silica nanoparticles because they may be used as a substrate capable of combining various types of functional groups, which is an advantage. However, in order to introduce various functional molecules into silica nanoparticles, a linker is essential. Thus, there is a need to develop a linker that is stable and capable of loading multiple functional molecules into one molecule.

Accordingly, the present inventors developed a methallylsilane-based linker compound that may stably modify the surface of silica nanoparticles and introduce two or more functional molecules through continuous bioorthogonal reactions in a one-pot process, and then created the present disclosure.

### DISCLOSURE OF THE INVENTION

### Technical Goals

An aspect of the present disclosure is to provide a methallylsilane-based linker compound and a method for producing the same.

Another aspect of the present disclosure is to provide silica nanoparticles having a surface modified with the linker compound and a method for producing the same.

A further aspect of the present disclosure is to provide a nanoprobe including the silica nanoparticles; and two or more functional molecules, and a method for manufacturing the same.

Another aspect of the present disclosure is to provide a composition for in vivo molecular imaging, including the nanoprobe.

A further aspect of the present disclosure is to provide a composition for theranostics including the nanoprobe.

However, the technical goals to be achieved by the present disclosure are not limited to those mentioned above, and other goals not mentioned will be clearly understood by one of ordinary skill in the art from the following description.

### Technical Solutions

In order to achieve the above mentioned goals, the present disclosure may provide a linker compound represented by the following [Chemical Formula 1]:

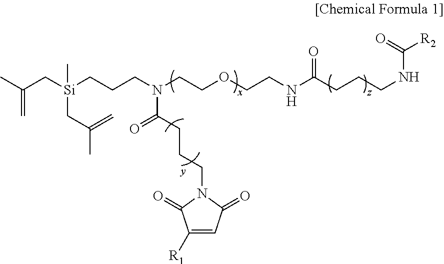

in Chemical Formula 1,
x, y, and z are the same or different from each other, and are each independently an integer from 1 to 10, preferably an integer from 1 to 5, and more preferably 2,
R₁ is a chained alkyl group of C₁-C₆,
R₂ is an —OR₃ group, —O(CH₂)R₄ group, or —NH(CH₂)ₙC(O)R₅ group (wherein n is an integer from 1 to 5),
R₃, R₄, and R₅ are the same or different from each other, and are each independently a cycloalkene group, a cycloalkyne group, a heterocycloalkene group, or a heterocycloalkyne group of C₃-C₂₀ (wherein the cycloalkene group, cycloalkyne group, heterocycloalkene group, or heterocycloalkyne group is capable of being unsubstituted or substituted with a chained alkyl group of C₁-C₆).

As an embodiment of the present disclosure, R₂ may be preferably

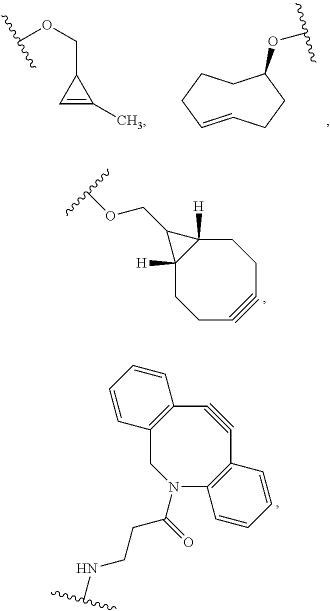

and the like, and more preferably,

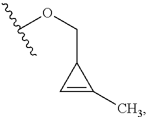 [SMILES: *OCC1C=C1C]

but is not limited thereto.

As another embodiment of the present disclosure, the linker compound may be a compound represented by the following [Chemical Formula 1-1]:

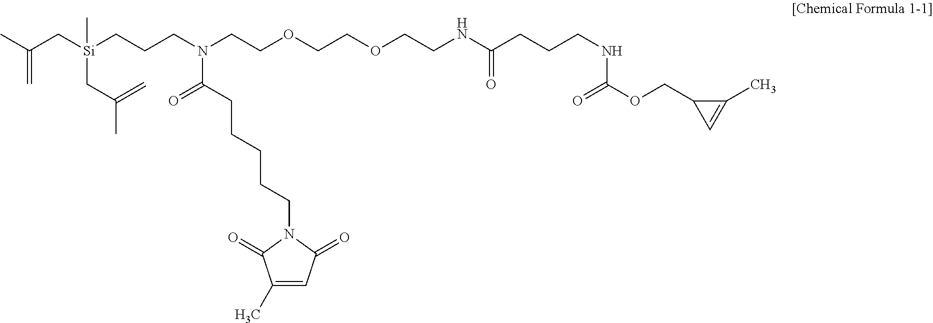 [SMILES: C=C(C)C[Si](C)(CCCN(CCOCCOCCNC(=O)CCCNC(=O)OCC1C=C1C)C(=O)CCCCCN1C(=O)C=C(C)C1=O)CC(=C)C]

As yet another embodiment of the present disclosure, the linker compound may be a bifunctional linker. That is, two functional molecules may be introduced into one linker molecule.

As yet another embodiment of the present disclosure, the functional molecule may be one or more selected from the group consisting of a fluorescent dye, a radioactive isotope, a magnetic substance, a ligand, a drug, and a combination thereof, but is not limited thereto.

In addition, the present disclosure may provide a method for producing a linker compound represented by [Chemical Formula 1], including reacting a compound represented by [Chemical Formula 2] with a compound represented by [Chemical Formula 3]:

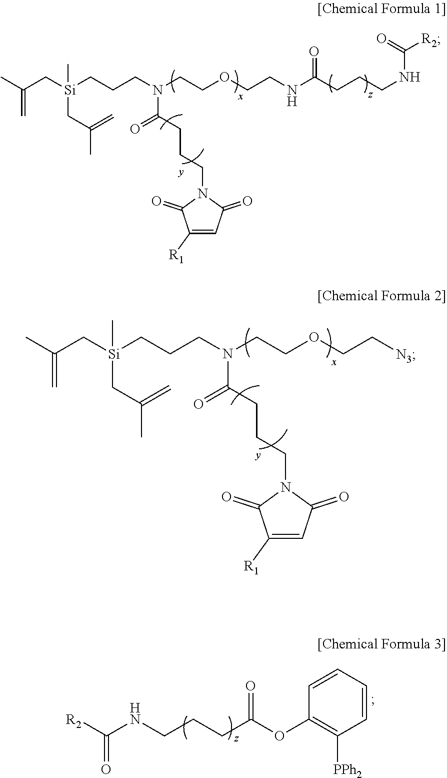

in Chemical Formulae 1 to 3,
x, y, and z are the same or different from each other, and are each independently an integer from 1 to 10, preferably an integer from 1 to 5, and more preferably 2,
R₁ is a chained alkyl group of C₁-C₆,
R₂ is an —OR₃ group, —O(CH₂)R₄ group, or —NH(CH₂)ₙC(O)R₅ group (wherein n is an integer from 1 to 5),
R₃, R₄, and R₅ are the same or different from each other, and are each independently a cycloalkene group, a cycloalkyne group, a heterocycloalkene group, or a heterocycloalkyne group of C₃-C₂₀ (wherein the cycloalkene group, cycloalkyne group, heterocycloalkene group, or heterocycloalkyne group is capable of being unsubstituted or substituted with a chained alkyl group of C₁-C₆).

Furthermore, the present disclosure may provide a silica nanoparticle, characterized in that a surface is modified with the linker compound.

In addition, the present disclosure may provide a method for producing silica nanoparticles into which a linker compound is introduced, the method including a step of treating silica nanoparticles with the linker compound of claim 1.

In addition, the present disclosure may provide a nanoprobe including the silica nanoparticles; and two or more functional molecules.

As an embodiment of the present disclosure, the functional molecule may be one or more selected from the group consisting of a fluorescent dye, a radioactive isotope, a magnetic substance, a ligand, a drug, and a combination thereof, but is not limited thereto.

As another embodiment of the present disclosure, the fluorescent dye may be one or more selected from the group consisting of FAM (6-carboxyfluorescein), DIG (Digoxigenin), FITC (fluorescein isothiocyanate), Texas Red, fluorescein, HEX (2′,4′,5′,7′-tetrachloro-6-carboxy-4,7-dichlorofluorescein), fluorescein chlorotriazinyl, rhodamine green, rhodamine red, tetramethyl rhodamine, oregon green, alexa fluor, JOE (6-Carboxy-4′,5′-Dichloro-2′,7′-Dimethoxyfluorescein), ROX (6-Carboxyl-XRhodamine), TET (Tetrachloro-Fluorescein), TRITC (tertramethylrodamine isothiocyanate), TAMRA (6-carboxytetramethyl-rhodamine), NED (N-(1-Naphthyl) ethylenediamine), thiadicarbocyanine, cyanine-based dye, BODIPY-based dye, coumarin-based dye, methylene blue, and a combination thereof, but is not limited thereto. Preferably, the fluorescent dye may be one or more a cyanine-based dye or a BODIPY-based dye selected from the group consisting of Cy3, Cy3.5, Cy5, Cy5.5, Cy7, Cy7.5, and a combination thereof, and more preferably Cy5.5.

As yet another embodiment of the present disclosure, the radioactive isotope may be one or more selected from the group consisting of ¹⁸F, ¹¹C, ¹³C, ¹³N, ¹⁵O, ⁶⁰Cu, ⁶⁴Cu, ⁶⁷Cu, ¹²⁴I, ⁶⁸Ga, ⁵²Fe, ⁵⁸Co, ³H, ¹⁴C, ³⁵S, ³²P, ¹³¹I, ⁵⁹Fe, ⁶⁰Co, ⁸⁹Sr, ⁹⁰Sr, ⁹⁰Y, ⁹⁹Mo, ¹³³Xe, ¹³⁷Cs, ¹⁵³Sm, ¹⁷⁷Lu, ¹⁸⁶Re, ¹²³I, ¹²⁵I, ²⁰¹Tl, ⁶⁷Ga, and a combination thereof, and more preferably ¹²⁵I, but is not limited to thereto.

As yet another embodiment of the present disclosure, the drug may be one or more anticancer agents selected from the group consisting of paclitaxel, docetaxel, doxorubicin, sorafenib, Vemurafenib, irinotecan, cisplatin, alpharadin, mitoxantrone, cyclophosphamide, vinblastine, carboplatin, actinomycin-D, etoposide, teniposide, bisantrene, homoharringtonine, Gleevec (STI-571), 5-fluorouracil, busulfan, chlorambucil, melphalan, nitrogen mustard, nitrosourea, and a combination thereof, but is not limited thereto.

Additionally, the present disclosure may provide a method for producing a nanoprobe including adding two or more functional molecules to the silica nanoparticles.

As an embodiment of the present disclosure, the method may further include the steps of adding acetonitrile and performing ultrasonic treatment after the addition step.

In addition, the present disclosure may provide a composition for in vivo molecular imaging, including the nanoprobe.

As an embodiment of the present disclosure, the imaging may be performed using one or more selected from the group consisting of fluorescence, bioluminescence, magnetic resonance imaging (MRI), computed tomography (CT), positron emission tomography (PET), single photon emission computed tomography (SPECT), and a combination thereof, but is not limited thereto.

Additionally, the present disclosure may provide a composition for theranostics, including the nanoprobe.

As an embodiment of the present disclosure, the composition may be capable of diagnosing cancer, cardiovascular disease, brain disease, etc., and treating them at the same time.

### Effects of the Invention

The present disclosure relates to a methallylsilane-based linker compound and a method for producing the same. The linker compound of the present disclosure may introduce two or more functional molecules into one molecule and may be covalently linked to the functional molecules, and thus, the linker compound may have very excellent stability.

According to one embodiment of the present disclosure, a surface of silica nanoparticles may be easily modified with the linker compound, and in particular, even when the surface of silica nanoparticles with a small size of 50 nanometers (nm) or less is modified, a particle aggregation phenomenon does not occur.

According to one embodiment of the present disclosure, it is possible to produce nanoprobes into which two or more desired functional molecules are selectively introduced, in a simple one-pot process through a continuous bioorthogonal reaction, by adding two or more functional molecules to the silica nanoparticles modified with the linker compound, while not disturbing a biological system.

According to one embodiment of the present disclosure, the nanoprobe may incorporate a targeting ligand capable of delivering a drug to a specific position, a fluorescent dye for molecular imaging, a radioactive isotope, a magnetic substance, a drug for treatment, a polyethylene glycol (PEG) capable of increasing biocompatibility, and the like, and thus may be utilized in in vivo molecular imaging, such as fluorescence, bioluminescence, magnetic resonance imaging (MRI), computed tomography (CT), positron emission tomography (PET), single photon emission computed tomography (SPECT), or theranostics, and the like.

In the present disclosure, when the silica nanoparticles modified with the linker compound are used as a support, the silica nanoparticles may be easily recovered from a reaction solvent as a heterogeneous catalyst in which two or more catalysts, and the like are added, and thus the silica nanoparticles may be recyclable after being used several times in specific organic reactions, such as an aldol reaction, a cycloaddition reaction, and the like.

The effects of the linker compound, silica nanoparticles, and nanoprobe according to an embodiment of the present disclosure are not limited to those mentioned above, and other effects not mentioned will be clearly understood by one of ordinary skill in the art from the following description.

### BRIEF DESCRIPTION OF DRAWINGS

FIG. 1 shows a process of synthesizing maleimide conjugated allylsilane (compound 6) and cyclopropene conjugated acid (compound 11). Here, reagents and conditions are as follows: (a) amine (compound 3), K₂CO₃ (2.0 equiv.), 80° C., 58%; (b) acid (compound 5), EDCI (1.2 equiv.), DMAP (1.0 equiv.), CH₂Cl, rt, 78%; (c) Me-C-TMS, Rh₂(OAc)₄ (0.01 equiv.), CH₂Cl₂, rt, 44%; (d) DIBAL (2.0 equiv.), Et₂O, 0° C., 85%; (e) i) CDI (1.1 equiv.), THF, rt; ii) MeO₂C(CH₂)₅NH₃Cl, Et₃N (2.0 equiv.), DMF, 50° C., 51%; (f) KOH (3.0 equiv.), MeOH/H₂O (4:1), 0° C.; (g) TBAF (3.0 equiv.), THF, rt, 95% (2 steps).

FIG. 2 shows a process of synthesizing allylsilane (compound 15) including a dual bioorthogonal linker through Staudinger ligation. Here, reagents and conditions are as follows: (a) Ph₂PH (1.0 equiv.), Pd(OAc)₂ (0.03 equiv.), NaOAc (1.0 equiv.), DMAc, 110° C., 71%; (b) Compound 11, EDCI (1.4 equiv.), DMAP (1.0 equiv.), DMAc, 110° C., 58%; (c) Compound 6, H₂O (3.0 equiv.), THF, 40° C., 52%.

FIG. 3 shows a synthesis and characteristics of silica nanoparticles (compound 20) modified with a linker compound (compound 15) according to an embodiment of the present disclosure; FIG. 3A shows a process of synthesizing the silica nanoparticles, FIG. 3B shows a DLS graph of the silica nanoparticles, and FIG. 3C shows an SEM image of a surface of the silica nanoparticles before and after modification of the linker compound.

FIG. 4 shows a process of synthesizing a nanoprobe (compound 22) into which a fluorescent dye and tetrazine are introduced according to an embodiment of the present disclosure.

FIG. 5 shows characteristics of the nanoprobe (compound 22) of the present disclosure; FIG. 5A shows a DLS graph and SEM image of the nanoprobe, FIG. 5B shows an excitation spectrum and emission spectrum of the nanoprobe, and FIG. 5C shows a UV absorption intensity at 535 nm of (i) silica nanoparticles@linker-BODIPY, silica nanoparticles@linker-BODIPY-Tz, and tetrazine, for the cycloaddition reaction of tetrazine (compound 18) in CH₃CN.

FIG. 6 shows a process of synthesizing a nanoprobe (compound 28) into which a fluorescent dye and a radioactive isotope are introduced according to an embodiment of the present disclosure; FIG. 6A shows a process of synthesizing tetrazine (compound 26) labeled with ¹²⁵I, and FIG. 6B shows a process of one-pot synthesizing a nanoprobe in which Cy5.5 dye (compound 27) and tetrazine (compound 26) labeled with ¹²⁵I are introduced into the silica nanoparticles (compound 20).

FIG. 7 shows results of positron emission tomography (PET) and fluorescence biodistribution imaging using a nanoprobe (compound 28) according to an embodiment of the present disclosure in normal mice; FIG. 7A shows a three-dimensional PET/CT image of the whole body, FIG. 7B shows a cross-sectional PET/CT image of the lung (top), liver (middle), and spleen (bottom), FIG. 7C shows results of quantitative analysis of radioactivity for each organ, and FIG. 7D shows fluorescence images.

### BEST MODE FOR CARRYING OUT THE INVENTION

The present inventors developed a methallylsilane-based linker compound that may easily modify the surface of silica nanoparticles and stably introduce two or more functional molecules via covalent bonds, and then created the present disclosure.

More specifically, silica nanoparticles were modified with the linker compounds, and then a fluorescent dye and a radioisotope were introduced as functional molecules through bioorthogonal reactions to verify the bimolecular imaging effect.

Accordingly, the present disclosure provides a linker compound represented by the following [Chemical Formula 1]:

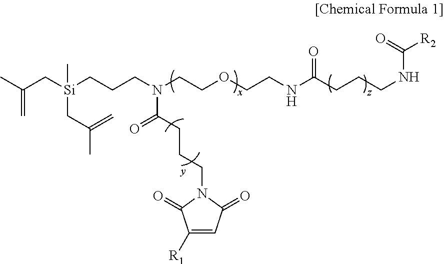

wherein, x, y, and z are the same or different from each other, and are each independently an integer from 1 to 10, preferably an integer from 1 to 5, and more preferably 2; R₁ is a chained alkyl group of C₁-C₆; R₂ is —OR₃ group, —O(CH₂)R₄ group, or —NH(CH₂)ₙC(O)R₅ group, wherein n is an integer from 1 to 5; R₃, R₄, and R₅ may be the same or different from each other, and each independently may be a cycloalkene group, a cycloalkyne group, a heterocycloalkene group, or a heterocycloalkyne group of C₃-C₂₀, wherein the cycloalkene group, cycloalkyne group, heterocycloalkene group, or heterocycloalkyne group may be unsubstituted or substituted with a chained alkyl group of C₁-C₆.

As used herein, the term “substitution” refers to a reaction in which an atom or atomic group contained in a molecule of a compound is replaced with another atom or atomic group.

As used herein, the term “chained alkyl group” refers to a group derived from a straight-chain or branched-chain saturated aliphatic hydrocarbon having a specified number of carbon atoms and having at least one valency. Examples of such alkyl groups include, but are not limited to, methyl, ethyl, propyl, isopropyl, butyl, isobutyl, 2-butyl, 3-butyl, pentyl, n-hexyl, etc.

As used herein, the term “cycloalkene group”, also called a cyclic alkene group, refers to a ring group in which all ring members are carbon and have one or more double bonds (but are not aromatic). The term “cycloalkyne group” refers to a ring group, also called a cyclic alkyne group, in which all ring members are carbon and have one or more triple bonds. The terms “heterocycloalkene group” and “heterocycloalkyne group” respectively refer to a cycloalkene group and a cycloalkyne group containing at least one of N, O, or S heteroatom. Each of the cycloalkene group, cycloalkyne group, heterocycloalkene group, or heterocycloalkyne group includes one or more ring structures, and may be, for example, a single ring, double ring, triple ring, etc.

Additionally, the present disclosure provides silica nanoparticles having a surface modified with the linker compound.

In addition, the present disclosure provides a nanoprobe including the silica nanoparticles; and two or more functional molecules.

As used herein, the term “linker” refers to a bond, compound, or molecule that connects a nanoparticle and a functional molecule, and the term “bifunctional linker” refers to a linker on one molecule, the linker being able to bind to two functional molecules.

As used herein, the term “nanoparticle” refers to a particle that is less than 1 micrometer in size. The nanoparticle may have an average particle size, which is the average longest dimension of the particle, of 1000 nanometers or less, 500 nanometers or less, 200 nanometers or less, 100 nanometers or less, 75 nanometers or less, 50 nanometers or less, 40 nanometers or less, 25 nanometers or less, or 20 nanometers or less. As used herein, the term “average particle size” refers to the average size of a single, unagglomerated and non-aggregated nanoparticle, which may be determined mainly by using a transmission electron microscope or various light scattering methods (e.g., laser diffraction).

As used herein, the term “silica” refers to an amorphous silicon dioxide (SiO₂), and the term “silica nanoparticle” refers to a nanoparticle having a silica surface. That is, “silica nanoparticle” may mean a nanoparticle composed entirely of silica, as well as a nanoparticle containing an organic core or other inorganic cores (e.g., metal oxides) with a silica surface.

As used herein, the term “functional molecule” refers to any molecule that has utility within a cell. Examples of functional molecules include a targeting ligand capable of delivering a drug to a specific position, a fluorescent dye for molecular imaging, a radioactive isotope, a magnetic substance, a drug for treatment, and the like, but are not limited thereto.

As used herein, the term “a fluorescent dye” refers to a dye that emits fluorescence with visible light or ultraviolet light, more specifically, a fluorescently labeled dye for labeling silica nanoparticles or the like. The fluorescent dye may be used to directly label biomolecules through commonly used chemical reactions.

Examples of the fluorescent dye include FAM (6-carboxyfluorescein), DIG (Digoxigenin), FITC (fluorescein isothiocyanate), Texas Red, fluorescein, HEX (2′,4′,5′,7′-tetrachloro-6-carboxy-4,7-dichlorofluorescein), fluorescein chlorotriazinyl, rhodamine green, rhodamine red, tetramethyl rhodamine, oregon green, alexa fluor, JOE (6-Carboxy-4′,5′-Dichloro-2′,7′-Dimethoxyfluorescein), ROX (6-Carboxyl-XRhodamine), TET (Tetrachloro-Fluorescein), TRITC (tertramethylrodamine isothiocyanate), TAMRA (6-carboxytetramethyl-rhodamine), NED (N-(1-Naphthyl) ethylenediamine), thiadicarbocyanine, cyanine-based dye, BODIPY-based dye, coumarin-based dye, methylene blue, etc., but are not limited thereto.

As used herein, the term “radioactive isotope (RI)” refers to an isotope that emits radiation and decays into a stable form, also called a radionuclide. The nanoprobe according to an embodiment of the present disclosure may contain a radioactive isotope, in which case, the biodistribution in the body may be checked by tracking the emitted positrons via positron emission tomography (PET) in the body.

Examples of the radioactive isotope include ¹⁸F, ¹¹C, ¹³C, ¹³N, ¹⁵O, ⁶⁰Cu, ⁶⁴Cu, ⁶⁷Cu, ¹²⁴I, ⁶⁸Ga, ⁵²Fe, ⁵⁸Co, ³H, ¹⁴C, ³⁵S, ³²P, ¹³¹I, ⁵⁹Fe, ⁶⁰Co, ⁸⁹Sr, ⁹⁰Sr, ⁹⁰Y, ⁹⁹Mo, ¹³³Xe, ¹³⁷Cs, ¹⁵³Sm, ¹⁷⁷Lu, ¹⁸⁶Re, ¹²³I, ¹²⁵I, ²⁰¹Tl, ⁶⁷Ga, and the like, but are not limited to thereto.

As used herein, the term “a magnetic substance” refers to a substance that is magnetized and behaves like a magnet when subjected to a magnetic field. An MRI contrast agent containing a magnetic substance is commonly used when magnetic resonance imaging (MRI) is performed. The contrast agent serves to increase the resolution of MRI images, by increasing the number of hydrogen atoms that axis changes within blood vessels. In particular, the resolution and contrast of images of soft tissues such as muscles and ligaments are better, which is advantageous in the process of diagnosing and monitoring the progress of diseases such as cerebral infarction using brain nervous system imaging.

As used herein, the term “drug” refers to any substance capable of preventing or treating a disease, the term “preventing” refers to any action that inhibits or delays the occurrence, spread, or recurrence of a disease, and the term “treating” means any action that ameliorates or beneficially changes the symptoms of a disease.

The nanoprobe of the present disclosure is capable of in vivo molecular imaging, and thus it may be utilized for theranostics.

As used herein, the term “in vivo molecular imaging” refers to the imaging of biological processes at the cellular or subcellular level in vivo to characterize and quantify them, also called biomolecular imaging. The images generated by these methods reflect the molecular metabolic pathways of cells, and especially reflect the disease process in the physiological environment of living organisms in disease models, and thus may be utilized for early diagnosis of a disease, drug development, anticancer treatment, monitoring for gene-stem cell therapy, etc.

The “in vivo molecular imaging” refers to one or more techniques selected from the group consisting of fluorescence, bioluminescence, magnetic resonance imaging (MRI), computed tomography (CT), positron emission tomography (PET), single photon emission computed tomography (SPECT), and a combination thereof, but is not limited thereto.

As used herein, the term “theranostics” is a combination of the words therapeutics and diagnostics, and refers to a technique for diagnosing a disease using a substance that targets a lesion while simultaneously delivering a drug only to the affected site to treat the disease.

The terminology used in this application is used only to describe a specific embodiment and is not intended to limit the invention. The singular form of a word may also have the meaning of the plural form of the word unless the context clearly dictates otherwise. In the present application, the terms “comprise” or “having” and the like refer to a feature, a number, a step, an operation, an element, a component or a combination thereof described in the specification, but do not preclude the possibility that other features, numbers, steps, operations, elements, components, or a combination thereof may be present or added.

Unless defined otherwise, all terms used herein, including technical or scientific terms, have the same meaning as is commonly understood by one of ordinary skill in the art to which this invention belongs. Terms defined in commonly used dictionaries should be interpreted as having a meaning consistent with the meaning that may be understood from the context of the relevant art and are not to be interpreted in an ideal or overly formal sense unless they are explicitly defined in the present application.

To describe the components of an embodiment of the present disclosure, terms such as first, second, A, B, (a), and (b) may be used. These terms are only for distinguishing one component from another component, and do not limit the nature, order or sequence of the components. If a component is described as being “linked”, “coupled” or “connected” to another component, that component may be directly linked or connected to that other component, but it will be understood that another component may be “linked”, “coupled” or “connected” between the components.

Hereinafter, embodiments will be described in detail with reference to the accompanying drawings. However, since various changes may be made to the embodiments, it will be understood that the descriptions of the embodiments do not limit or otherwise restrict the scope of the patent application. With respect to the embodiments, it should be understood that the scope of the rights shall include all modifications, equivalents and substitutes.

In the description with reference to the accompanying drawings, the same components are given the same reference numerals regardless of the drawing numerals, and any overlapping description of a component has been omitted. With respect to the description of an embodiment, in case where it was determined that including a detailed description of a related known technology may unnecessarily obscure the gist of the embodiment, such detailed description was omitted.

As the inventive concept allows for various changes and numerous embodiments, particular embodiments will be illustrated in the drawings and described in detail in the written description. However, such illustrations and descriptions are not intended to limit the present disclosure to specific embodiments, and should be understood as including all transformations, equivalents, and substitutes that may be included in the spirit and scope of the present disclosure. In the following description of the present disclosure, in case where it was determined that a detailed description of a related known technology may obscure the gist of the present disclosure, such detailed description was omitted.

### MODE FOR CARRYING OUT THE INVENTION

### Example 1. Preparation of Linker Compounds of the Invention

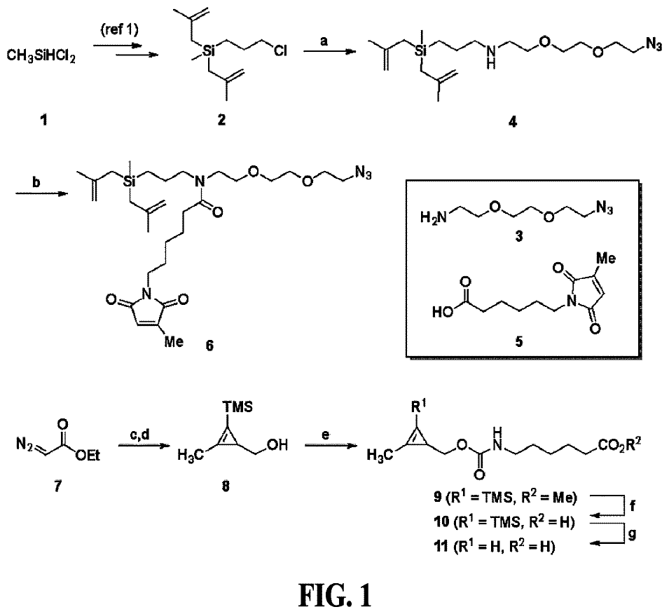

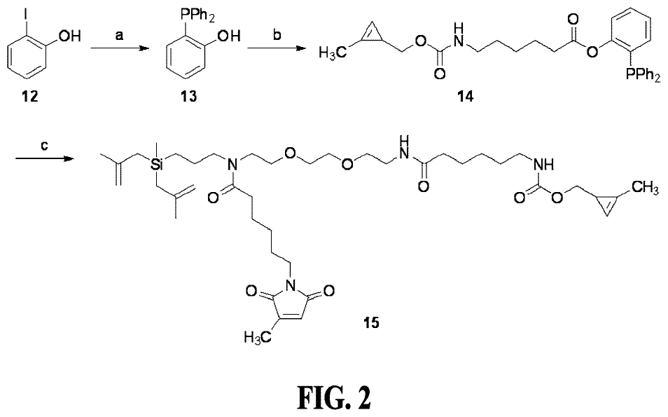

A method for preparing a linker compound according to an embodiment of the present disclosure is shown in FIGS. 1 and 2.

### Example 1.1. Synthesis of Maleimide-Conjugated Allylsilane (compound 6) 1.1.1. Preparation of 3-chloropropyldimethallylmethylsilane (Compound 2)

### (1) Preparation of 3-chloropropyldichloromethylsilane

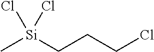

10 wt % H₂PtCl₆·xH₂O (4 mg, 0.01 mmol), 2-propanol (50 μL), and dichloromethylsilane (3 mL, 29 mmol) were added to a round bottom flask filled with argon and were stirred until homogeneous at room temperature. Diethyl ether (2.3 mL) was added. After the diethyl ether was added, the temperature was raised to 40° C. Allyl chloride (2.3 mL, 30 mmol) was slowly added and stirred at 60° C. for 12 hours. The mixture was cooled to room temperature. Thereafter, the mixture was placed under reduced pressure in a vacuum environment to remove the volatile constituents and to obtain a transparent 3-chloropropyldichloromethylsilane, which was used in the next step without further purification.

¹H-NMR (400 MHz, CDCl₃): δ 3.59 (t, J=6.5 Hz, 2H), 1.97 (m, 2H), 1.25 (m, 2H), 0.8 (s, 3H)

### (2) Preparation of 3-chloropropyldimethallylmethylsilane

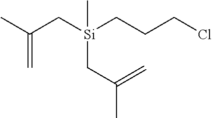

An excessive amount of 2-methallyl magnesium chloride solution was added to the 3-chloropropyldichloromethylsilane mixture, and then stirred at room temperature for 4 hours. Afterwards, ammonium chloride aqueous solution (30 mL) was added to terminate the reaction. The mixture was extracted using diethyl ether (3×20 mL) and washed with water and brine. The organic layer was dried over anhydrous MgSO₄, and then the solvent was removed under reduced pressure. The mixture was purified by silica gel column chromatography (hexane) to obtain the title compound (Compound 2, 1.6 g, 24.6%) as a pure clear liquid.

R_{f}=0.5, hexane, KMnO₄ development

¹H-NMR (400 MHz, CDCl₃): δ 4.62 (s, 2H), 6.50 (s, 2H), 3.50 (t, J=6.9 Hz, 2H), 1.81 (m, 2H), 1.72 (s, 6H), 1.59 (s, 4H), 0.71 (m, 2H), 0.07 (s, 3H)

### 1.1.2. Preparation of 2-(2-(2-azidoethoxy)ethoxy)ethane-1-amine (Compound 3)

### (1) Production of 1,2-bis(2-azidoethoxy)ethane

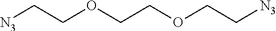 [SMILES: [N-]=[N+]=NCCOCCOCCN=[N+]=[N-]]

1,2-bis(2-chloroethoxy)ethane (1 g, 5.35 mmol) was dissolved in DMF (18 mL) in a round bottom flask filled with argon. Sodium azide (1.39 g, 21.38 mmol) was added and stirred at 90° C. for 12 hours. The mixture was cooled to room temperature. Thereafter, distilled water (10 mL) was added to terminate the reaction. The mixture was extracted using diethyl ether (3×30 mL) and washed with water and saline. The organic layer was dried over anhydrous MgSO₄, and then the solvent was removed under reduced pressure. The mixture was purified by silica gel column chromatography (hexane/EtOAc=1:1) to obtain the title compound (840 mg, 91%) as a pure clear liquid.

R_{f}=0.5, Hexane/EA=1:1 (v/v %), KMnO₄ development

¹H-NMR (400 MHz, CDCl₃): δ 3.72 (t, J=4.4 Hz, 8H), 3.42 (t, J=5.0 Hz, 4H)

### (2) Preparation of 2-(2-(2-azidoethoxy)ethoxy)ethane-1-amine

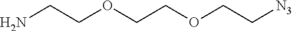 [SMILES: [N-]=[N+]=NCCOCCOCCN]

Triphenylphosphine (262 mg, 1 mmol) was slowly added to a solution of 1,2-bis(2-azidoethoxy)ethane (200 mg, 1 mmol) dissolved in EtOAc (3 mL) and HCl (1N, 2 mL). The mixture was stirred at room temperature for 12 hours. After the reaction (monitored by TLC) reached completion, HCl (1N) was added until the pH value reached 1, and then the mixture was extracted with EtOAc (3×15 mL). Afterwards, NaOH was added to the water layer until the pH value reached 14, then the mixture was extracted with dichloromethane (3×20 mL) and washed with water and brine. The organic layer was dried over anhydrous MgSO₄, and then the solvent was removed under reduced pressure to obtain the title compound (Compound 3, 104 mg, 60%) as a pure clear liquid.

R_{f}=0.25, CH₂Cl₂/MeOH=20:1 (v/v %), Ninhydrin development

¹H-NMR (400 MHz, CDCl₃): δ 3.68 (m, 6H), 3.54 (t, J=5.2 Hz, 2H), 3.41 (t, J=5.0 Hz, 2H), 2.89 (t, J=5.1 Hz, 2H)

### 1.1.3. Preparation of N-(2-(2-(2-azidoethoxy)ethoxy)ethyl)-3-(methylbis(2-methylallyl)silyl)propan-1-amine (Compound 4)

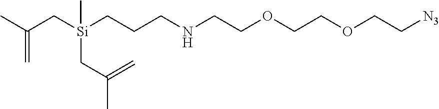 [SMILES: C=C(C)C[Si](C)(CCCNCCOCCOCCN=[N+]=[N-])CC(=C)C]

In a round bottom flask filled with argon, 2-(2-(2-azidoethoxy)ethoxy)ethane-1-amine (Compound 3, 800 mg, 4.59 mmol) was dissolved in acetonitrile (5.7 mL), and then 3-chloropropyldimethallylmethylsilane (Compound 2, 265 mg, 1.15 mmol) and K₂CO₃ (318 mg, 2.3 mmol) were added. The mixture was stirred at 80° C. for 72 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (CH₂Cl₂/MeOH=10:1) to obtain the title compound (Compound 4, 246 mg, 58%) as a pure clear liquid.

R_{f}=0.3, CH₂Cl₂/MeOH=10:1 (v/v %), KMnO₄ development

¹H-NMR (400 MHz, CDCl₃): δ 4.62 (s, 2H), 4.52 (s, 2H), 3.68 (m, 6H), 3.54 (t, J=5.2 Hz, 2H), 3.41 (t, J=5.0 Hz, 2H), 2.83 (t, J=5.2 Hz, 2H), 2.64 (t, J=7.3 Hz, 2H), 1.73 (s, 6H), 1.60 (s, 4H), 1.55 (m, 2H), 0.60 (m, 2H), 0.07 (s, 3H).; ¹³C-NMR (100 MHz; CDCl₃): δ 143.4, 108.9, 70.6, 70.4, 70.1, 53.2, 50.7, 49.1, 29.7, 25.6, 25.4, 24.1, 11.5, −4.5.; HRMS(ESI)(m/z): [M+H]⁺ calcd for C₁₈H₃₆N₄O₂Si 369.2685 found 369.2683.

### 1.1.4. Preparation of 6-(3-methyl-2,5-dioxo-2,5-dihydro-1H-pyrrol-1-yl)hexanoic acid (Compound 5)

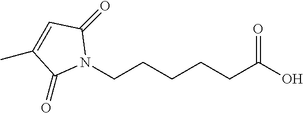 [SMILES: CC1=CC(=O)N(CCCCCC(=O)O)C1=O]

In a round bottom flask filled with argon, 6-aminohexanoic acid (1 g, 7.62 mmol) was dissolved in acetic acid (6.1 mL), and then citraconic anhydride (0.75 mL, 8.38 mmol) was added. The mixture was stirred at 100° C. for 4 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (hexane/EtOAc=1:1) to obtain the title compound (Compound 5, 1.5 g, 90%) as a pure white solid.

R_{f}=0.5, hexane/EtOAc=1:1 (v/v %), KMnO₄ development

¹H-NMR (400 MHz, CDCl₃): δ 6.33 (d, J=1.8 Hz, 2H), 3.52 (t, J=7.2 Hz, 2H), 2.37 (t, J=7.4 Hz, 2H), 2.10 (d, J=1.8 Hz, 3H), 1.65 (m, 4H), 1.36 (m, 2H).

### 1.1.5. Preparation of N-(2-(2-(2-azidoethoxy)ethoxy)ethyl)-6-(3-methyl-2,5-dioxo-2,5-dihydro-1H-pyrrol-1-yl)-N-(3-(Methylbis(2-methylallyl)silyl)propyl)hexanamide (Compound 6)

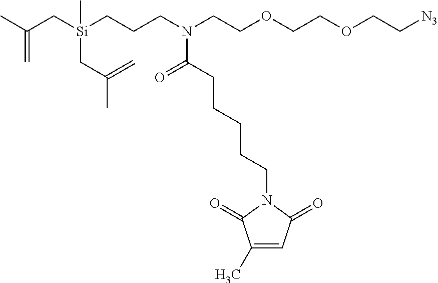 [SMILES: C=C(C)C[Si](C)(CCCN(CCOCCOCCN=[N+]=[N-])C(=O)CCCCCN1C(=O)C=C(C)C1=O)CC(=C)C]

In a round bottom flask filled with argon, N-(2-(2-(2-azidoethoxy)ethoxy)ethyl)-3-(methylbis(2-methylallyl)silyl)propan-1-amine (compound 4, 535 mg, 1.49 mmol) was dissolved in dichloromethane (15 mL), and then 6-(3-methyl-2,5-dioxo-2,5-dihydro-1H-pyrrol-1-yl)hexanoic acid (Compound 5, 370 mg, 1.64 mmol), 4-dimethylaminopyridine (184 mg, 1.49 mmol), and 1-ethyl-3-(3-dimethylaminopropyl)carboimide (320 μL, 1.80 mmol) were added. The mixture was stirred at room temperature for 1 hour. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (hexane/EtOAc=1:2) to obtain the title compound (Compound 6, Chemical Formula 2-1, 677 g, 78%) as a pure clear liquid.

R_{f}=0.3, hexane/EtOAc=1:2 (v/v %), KMnO₄ development

¹H-NMR (400 MHz, CDCl₃): δ 6.32 (d, J=1.8 Hz, 1H), 4.63 (m, 2H), 4.51 (m, 2H), 3.65 (m, 8H), 3.51 (m, 4H), 3.40 (m, 2H), 3.30 (m, 2H), 2.32 (m, 2H), 2.09 (d, J=1.8 Hz, 3H), 1.64 (m, 16H), 1.35 (m, 2H), 0.53 (m, 2H), 0.08 (m, 3H).; ¹³C-NMR (100 MHz; CDCl₃): δ 172.8, 172.6, 171.8, 170.9, 154.5, 143.2, 143.0, 127.2, 109.0, 108.8, 70.8, 70.7, 70.6, 70.3, 70.1, 70.0, 69.4, 69.4, 52.2, 50.6, 49.4, 47.5, 45.8, 37.8, 37.7, 32.9, 32.8, 28.5, 26.7, 26.6, 25.7, 25.6, 25.4, 24.9, 23.3, 21.8, 11.1, 11.0, 10.9, −4.4, −4.5.; HRMS(ESI)(m/z): [M+H]⁺ calcd for C₂₉H₄₉N₅O₅SiNa 598.3400. found 598.3400.

### Example 1.2. Synthesis of cyclopropene conjugated acid (Compound 14)

### 1.2.1. Preparation of (2-methyl-3-(trimethylsilyl)cycloprop-2-ene-1-yl)methanol (Compound 8)

### (1) Preparation of ethyl 2-methyl-3-(trimethylsilyl)cycloprop-2-ene-1-carboxylate

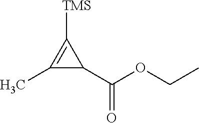

In a round bottom flask filled with argon, Rh₂(OAc)₄ (20 mg, 0.04 mmol) and trimethylsilylacetylene (1.3 mL, 8.76 mmol) was dissolved in dichloromethane (17 mL), and then ethyldiazoacetate (Compound 7, 500 mg, 4.38 mmol) was slowly added at a rate of 1.0 mL/h. The mixture was stirred at room temperature for 1 hour. After the reaction (monitored by TLC) reached completion, the solvent was removed under reduced pressure immediately without a separate extraction process being performed. The mixture was purified by silica gel column chromatography (hexane/EtOAc=15:1) to obtain the title compound (439 g, 50.5%) as a pure clear liquid.

R_{f}=0.4, hexane/EtOAc=10:1 (v/v %), KMnO₄ development

¹H-NMR (400 MHz, CDCl₃) δ 4.06 (m, 2H), 2.15 (s, 3H), 1.93 (s, 1H), 1.19 (t, J=7.1 Hz, 3H), 0.15 (s, 9H).

### (2) Preparation of (2-methyl-3-(trimethylsilyl)cycloprop-2-ene-1-yl)methanol

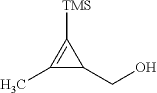

In a round bottom flask filled with argon, ethyl 2-methyl-3-(trimethylsilyl)cycloprop-2-ene-1-carboxylate (770 mg, 3.88 mmol) was dissolved in diethyl ether (20 mL), and then 1.0 M DIBAL toluene solution (8 mL, 7.7 mmol) was slowly added at 0° C. and stirred for 2 hours. After the reaction (monitored by TLC) reached completion, aqueous potassium sodium tartrate solution (10 mL) was added to terminate the reaction. The mixture was extracted using diethyl ether (3×15 mL) and washed with water and brine. The organic layer was dried over anhydrous MgSO₄, and then the solvent was removed under reduced pressure. The mixture was purified by silica gel column chromatography (hexane/EtOAc=5:1) to obtain the title compound (Compound 8, 677 g, 78%) as a pure clear liquid.

### 1.2.2. Preparation of 6-((((2-methylcycloprop-2-ene-1-yl)methoxy)carbonyl)amino)hexanoic acid (Compound 11)

### (1) Preparation of 6-aminoheptanoic acid methyl ester hydrochloride

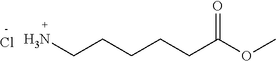

In a round bottom flask filled with argon, thionyl chloride (1.5 mL, 19.8 mmol) was slowly added to methanol (7.6 mL) at 0° C. After 5 minutes, 6-aminocaproic acid (1 g, 7.6 mmol) was added. The mixture was stirred at room temperature for 3 hours. Without further purification, the solution was evaporated under reduced pressure in a vacuum environment to obtain the title compound (1.37 g, 99%) as a pure yellow solid.

¹H-NMR (400 MHz, D2O): δ 3.61 (s, 3H), 2.91 (t, J=7.5 Hz, 2H), 2.34 (t, J=7.4 Hz, 2H), 1.57 (m, 4H), 1.32 (q, J=7.9 Hz, 2H).

### (2) Preparation of methyl 6-((((2-methyl-3-(trimethylsilyl)cycloprop-2-ene-1-yl)methoxy)carbonyl)amino)hexanoate (Compound 9)

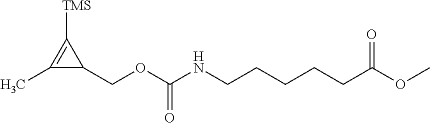

In a round bottom flask filled with argon, (2-methyl-3-(trimethylsilyl)cycloprop-2-ene-1-yl)methanol (Compound 8, 100 mg, 0.64 mmol) was dissolved in THE (4.2 mL), and then carbonyldiimidazole (115 mg, 0.7 mmol) was added. The mixture was stirred at room temperature for 3 hours. After the reaction (monitored by TLC) reached completion, the solvent was removed under reduced pressure. The mixture was used in the next step without further purification. The mixture was dissolved in DMF (1.3 mL), and then 6-aminoheptanoic acid methyl ester hydrochloride (128 mg, 0.70 mmol) and triethylamine (0.18 mL, 1.28 mmol) were added. The mixture was stirred at 50° C. for 12 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (hexane/EtOAc=2:1) to obtain the title compound (Compound 9, 73 g, 35%) as a pure clear liquid.

R_{f}=0.3, hexane/EtOAc=2:1 (v/v %), KMnO₄ development

¹H-NMR (400 MHz, CDCl₃): δ 4.64 (s, 1H), 3.91 (m, 2H), 3.68 (s, 3H), 3.19 (q, J=6.7 Hz, 2H), 2.33 (t, J=7.4 Hz, 2H), 2.20 (s, 3H), 1.70 (m, 3H), 1.55 (m, 3H), 1.39 (m, 2H), 0.17 (s, 9H).; ¹³C-NMR (100 MHz; CDCl₃): δ 174.0, 157.0, 134.6, 111.0, 73.3, 51.5, 40.7, 33.9, 29.8, 26.2, 24.6, 18.6, 13.3, −1.2.; LRMS(ESI)(m/z): [M+H]⁺ calcd for C₁₆H₂₉NNaO₄Si 350.2 found 350.1.

### (3) Preparation of 6-((((2-methylcycloprop-2-ene-1-yl)methoxy)carbonyl)amino)hexanoic acid

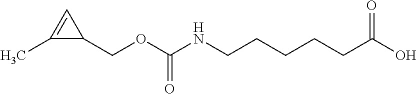 [SMILES: CC1=CC1COC(=O)NCCCCCC(=O)O]

Methyl 6-((((2-methyl-3-(trimethylsilyl)cycloprop-2-ene-1-yl)methoxy)carbonyl)amino)hexanoate (Compound 9, 216 mg, 0.66 mmol) was dissolved in methanol (2.2 mL). Potassium hydroxide (111 mg, 1.98 mmol) was dissolved in distilled water (0.6 mL) and added to the reaction mixture at 0° C. The mixture was stirred for 3 hours. After the reaction (monitored by TLC) reached completion, 1 M (1 mL) HCl was added, the mixture was extracted using dichloromethane (3×10 mL), and washed with water and brine. The organic layer was dried over anhydrous MgSO₄, and then the solvent was removed under reduced pressure. The mixture was used in the next step without further purification.

R_{f}=0.2, hexane/EtOAc=1:2 (v/v %), KMnO₄ development

The above mixture was dissolved in THE (6.6 mL), and then TBAF (2 mL) was added. The mixture was stirred at room temperature for 2 hours. After the reaction (monitored by TLC) reached completion, the mixture was extracted using dichloromethane (3×10 mL), and washed with water and brine. The organic layer was dried over anhydrous MgSO₄, and then the solvent was removed under reduced pressure. Without further purification, the title compound (Compound 11, 110 mg, 74%) was obtained as a clear liquid.

R_{f}=0.2, hexane/EtOAc=1:3 (v/v %), KMnO₄ development

¹H-NMR (400 MHz, CDCl₃): δ 6.58 (s, 1H), 4.70 (s, 1H), 3.94 (d, J=4.2 Hz, 2H), 3.20 (d, J=4.0 Hz, 2H), 2.38 (t, J=7.4 Hz, 2H), 2.15 (s, 3H), 1.67 (m, 3H), 1.54 (m, 2H), 1.40 (m, 2H)

### 1.2.3. Preparation of 2-(diphenylphosphanyl)phenol (Compound 13)

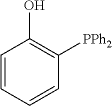

DMAc (15 mL) was added to a round bottom flask filled with argon, and the oxygen in the solvent was removed with argon gas. 2-iodophenol (Compound 12, 1 g, 4.54 mmol) was dissolved in a solvent, and then diphenylphosphine (0.8 mL, 4.54 mmol), palladium (II) acetate (10 mg, 0.045 mmol), and sodium acetate (400 mg, 4.99 mmol) were added. The mixture was stirred at 110° C. for 2 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (hexane/CH₂Cl₂=1:2) to obtain the title compound (Compound 13, 800 mg, 63%) as a pure white solid.

R_{f}=0.3, hexane/CH₂Cl₂=1:2, KMnO₄ development

¹H-NMR (400 MHz, CDCl₃): δ 7.37 (m, 11H), 7.01 (dt, J=5.6 Hz, 13.1 Hz, 2H), 6.92 (t, J=7.4 Hz, 1H), 6.67 (br, 1H)

### 1.2.4. Preparation of 2-(diphenylphosphanyl)phenyl 6-((((2-methylcycloprop-2-ene-1-yl)methoxy)carbonyl)amino)hexanoate (Compound 14)

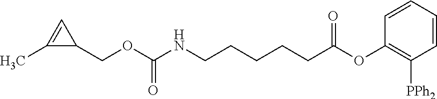

In a round bottom flask filled with argon, 6-((((2-methylcycloprop-2-ene-1-yl)methoxy)carbonyl)amino)hexanoic acid (Compound 11, 175 mg, 0.73 mmol) was dissolved in CH₂Cl₂ (6.6 mL), and then 2-(diphenylphosphanyl)phenol (Compound 13, 202 mg, 0.73 mmol), 1-ethyl-3-(3-dimethylaminopropyl)carbodiimide (140 μL, 0.79 mmol), and 4-dimethylaminopyridine (80 mg, 0.66 mmol) were added. The mixture was stirred at room temperature for 2 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (hexane/EtOAc=3:1) to obtain the title compound (Compound 14, Chemical Formula 3-1, 677 mg, 78%) as a pure white liquid.

R_{f}=0.3, hexane/EtOAc=3:1 (v/v %), KMnO₄ development

¹H-NMR (400 MHz, CDCl₃): δ 7.36 (m, 11H), 7.16 (dd, J=5.4, 11.5 Hz, 2H), 6.82 (m, 1H), 6.58 (s, 1H), 4.65 (s, 1H), 3.95 (d, J=4.6 Hz, 2H), 3.16 (q, J=6.2 Hz, 2H), 2.28 (t, J=7.4 Hz, 2H), 2.15 (d, J=0.8 Hz, 3H), 1.66 (t, J=4.6 Hz, 1H), 1.34 (m, 4H), 1.29 (q, J=5.2 Hz, 2H).; ¹³C-NMR (100 MHz; CDCl₃): δ 171.3, 156.9, 152.8, 152.7, 135.7, 135.6, 134.1, 133.9, 133.7, 130.2, 130.1, 129.90 129.0, 128.6, 128.6, 126.1, 22.6, 120.8, 102.2, 72.1, 40.7, 33.8, 29.7, 26.1, 24.1, 17.3, 11.7.; HRMS(ESI)(m/z): [M+H]⁺ calcd for C₃₀H₃₂NO₄PNa 524.1966 found 524.1964.

### Example 1.3. Synthesis of Linker Compound Containing Methallylsilane (Compound 15)

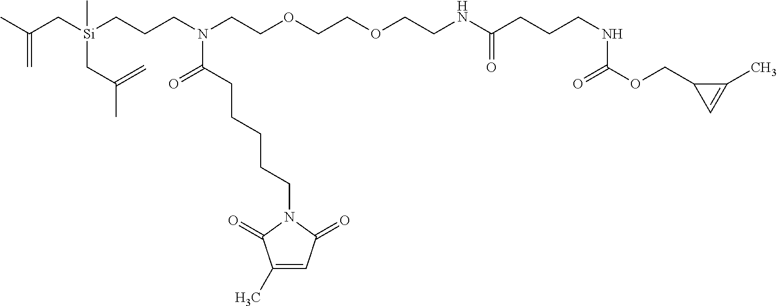 [SMILES: C=C(C)C[Si](C)(CCCN(CCOCCOCCNC(=O)CCCNC(=O)OCC1C=C1C)C(=O)CCCCCN1C(=O)C=C(C)C1=O)CC(=C)C]

In a round bottom flask filled with argon, N-(2-(2-(2-azidoethoxy)ethoxy)ethyl)-6-(3-methyl-2,5-dioxo-2,5-dihydro-1H-pyrrol-1-yl)-N-(3-(methylbis(2-methylallyl)silyl)propyl)hexanamide (Compound 6, Chemical Formula 2-1, 145 mg, 0.28 mmol) was dissolved in THE (6.6 mL), and then 2-(diphenylphosphanyl)phenyl 6-(((((2-methylcycloprop-2-ene-1-yl)methoxy)carbonyl)amino)hexanoate (Compound 14, Chemical Formula 3-1, 165 mg, 0.28 mmol) was added. The mixture was stirred at 40° C. for 12 hours. After the reaction (monitored by TLC) reached completion, distilled water (100 μL) was added and the mixture was stirred at room temperature for an additional 3 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (CH₂Cl₂/MeOH=20:1) to obtain (2-methylcycloprop-2-ene-1-yl)methyl(2,4-dimethyl-8-(6-(3-methyl-2,5-dioxo-2,5-dihydro-1H-pyrrol-1-yl))hexanoyl)-4-(2-methylallyl)-18-oxo-11,14-dioxa-8,17-diaza-4-silatricos-1-ene-23-yl)carbamate (Compound 15, Chemical Formula 1-1, 114 mg, 51%) as a pure white liquid.

R_{f}=0.3, CH₂Cl₂/MeOH=20:1 (v/v %), KMnO₄ development

¹H-NMR (400 MHz, CDCl₃): δ 6.58 (s, 1H), 6.32 (d, J=1.7 Hz, 1H), 6.15 (m, 1H), 4.79 (s, 1H), 4.64 (s, 2H), 4.51 (s, 2H), 3.93 (d, J=4.7 Hz, 2H), 3.57 (m, 14H), 3.30 (m, 2H), 3.18 (q, J=6.5 Hz, 2H), 2.33 (m, 2H), 2.23 (m, 2H), 2.15 (s, 3H), 2.09 (d, J=1.6 Hz, 3H), 1.74 (s, 6H), 1.61 (s, 15H), 1.35 (m, 4H), 0.54 (m, 2H), 0.08 (m, 3H).; ¹³C-NMR (100 MHz; CDCl₃): δ 173.0, 172.7, 171.9, 170.94 157.1, 145.5, 143.2, 143.0, 127.2, 120.8, 109.1, 108.9, 102.2, 72.1, 70.9, 70.5, 70.2, 70.0, 69.1, 70.0, 53.4, 52.3, 49.2, 47.5, 45.8, 40.8, 39.1, 37.83, 37.8, 36.4, 33.0, 32.9, 29.8, 28.5, 26.7, 26.6, 26.4, 25.7, 25.6, 25.49 25.3, 24.9, 23.4, 21.9, 17.3, 11.7, 11.2, 11.1, 11.0, −4.3, −4.4.; HRMS(ESI)(m/z): [M+H]⁺ calcd for C₄₁H₆₈N₄O₈SiNa 795.4704 found 795.4702.

### Example 2. Preparation of Modified Silica Nanoparticles of the Invention

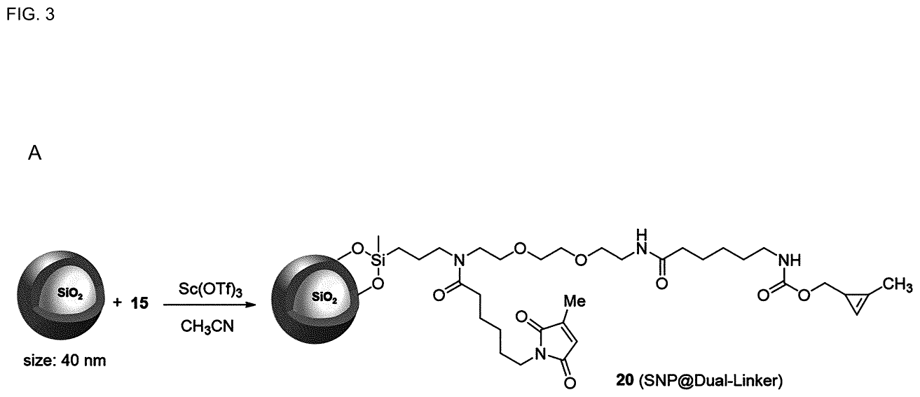

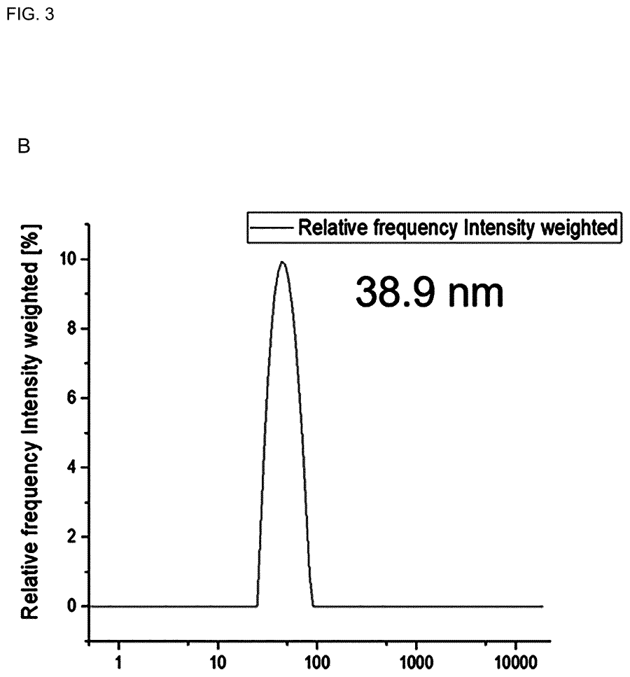

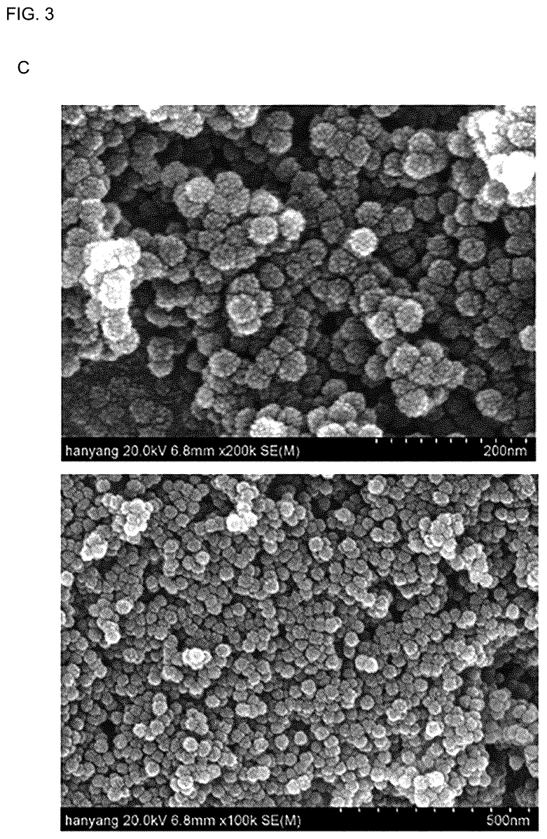

A method for producing silica nanoparticles modified with a linker compound according to an embodiment of the present disclosure is shown in FIG. 3a.

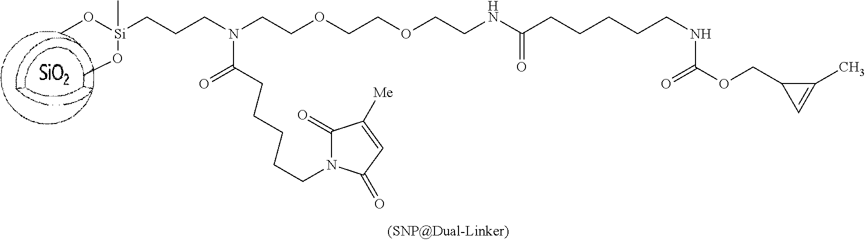

The bifunctional linker (Compound 15, Chemical Formula 1-1, 30 mg, 0.039 mmol) prepared during the process of Example 1 was dissolved in silica nanoparticle acetonitrile solution (20 mg/6 mL), Sc(OTf)₃ (1 mg, 0.001 mmol) was added, the mixture was stirred at room temperature for 90 minutes, then transferred to a 1 mL plastic vial, centrifugation was performed, the supernatant was discarded and fresh distilled water (1 mL each) was added, and ultrasonication was performed at maximum intensity for 10 minutes. The above described process was repeated three times. Afterwards, the same process was performed with acetonitrile. TLC was used to confirm whether there was any remaining bifunctional linker.

Size and shape were characterized using dynamic light scattering (DLS) and SEM (FIGS. 3b and 3c). The results showed that the size of the silica nanoparticles (38.9 nm) remained consistent without particle aggregation.

### Example 3. Preparation of Nanoprobe of the Invention

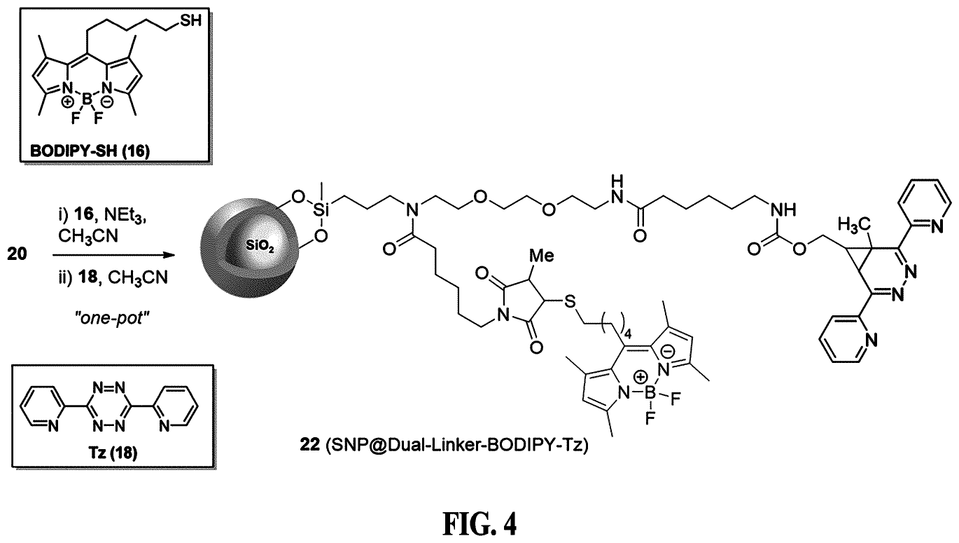

A method for producing a nanoprobe into which a fluorescent dye and tetrazine are introduced according to an embodiment of the present disclosure is shown in FIG. 4.

### Example 3.1. Synthesis of BODIPY Fluorescent Dye (Compound 16)

### 3.1.1. Preparation of 8-(5-bromopentyl)-4,4-difluoro-1,3,5,7-tetramethyl-4-bora-3a,4a-diaza-s-indacene

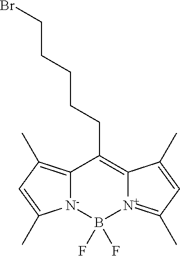

To a solution of 6-bromohexanoyl chloride (0.717 mL, 4.68 mmol) in CH₂Cl₂ (46 mL) at 0° C., was added 2,4-dimethylpyrrole (1.1 mL, 10.3 mmol). The reaction mixture was stirred at room temperature for 7 hours. Then, the mixture was cooled to 0° C. and Et₃N (1.96 mL, 14 mmol) was added. After 30 minutes, boron trifluoride etherate (2.89 mL, 23.4 mmol) was added and the mixture was stirred at room temperature for 6 hours. The reaction mixture was quenched with water (10 mL), extracted with CH₂Cl₂ (3×30 mL) and washed with brine. The combined organic layers were dried over MgSO₄ and concentrated under reduced pressure. The crude residue was purified by flash silica gel column chromatography to obtain the title compound as an orange solid (591 mg, 32%).

R_{f}=0.3, n-hexane/Dichloromethane=1:1 (v/v %).

¹H-NMR (400 MHz, CDCl₃): δ 6.08 (s, 2H), 3.46 (t, J=6.5 Hz, 2H), 2.98 (m, 2H), 2.53 (s, 6H), 2.44 (s, 6H), 1.95 (t, J=6.5 Hz, 2H), 1.68 (m, 4H).

### 3.1.2. Preparation of S-(5-(5,5-difluoro-1,3,7,9-tetramethyl-5H-414,514-dipyrrolo[1,2-c:2′,1′-f][1,3,2]diazaborinin-10-yl)pentyl)ethanethioate

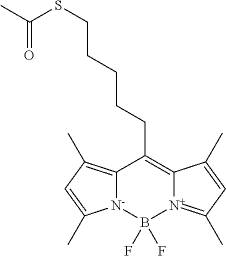

8-(5-bromopentyl)-4,4-difluoro-1,3,5,7-tetramethyl-4-bora-3a,4a-diaza-s-indacene (500 mg, 1.26 mmol) was dissolved in acetone (25 mL) and potassium thioacetate (173 mg, 1.51 mmol) was added. The solution was stirred under reflux for 3 hours. The mixture was extracted with DCM. The organic layer was dried over MgSO₄ and concentrated in a vacuum to obtain the title compound as an orange residue (480 mg, 97%), which was used in the next step without further purification.

R_{f}=0.4, n-hexane/Dichloromethane=1:1 (v/v %).

¹H-NMR (400 MHz, CDCl₃): δ 6.07 (s, 2H), 2.94 (m, 4H), 2.53 (s, 6H), 2.43 (s, 6H), 2.36 (s, 3H), 1.64 (m, 6H), 1.68 (m, 4H).

### 3.1.3. Preparation of 5-(5,5-difluoro-1,3,7,9-tetramethyl-5H-414,514-dipyrrolo[1,2-c:2′,1′-f][1,3,2]diazaborinin-10-yl)pentane-1-thiol (BODIPY-thiol, Compound 16)

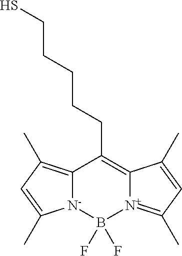

S-(5-(5,5-difluoro-1,3,7,9-tetramethyl-5H-414,514-dipyrrolo[1,2-c:2′,1′-f][1,3,2]diazaborinin-10-yl)pentyl)ethanethioate (300 mg, 0.76 mmol) was suspended in MeOH (15 mL) and degassed using argon gas for about 1 hour. After 1 hour, potassium carbonate (158 mg, 1.15 mmol) was added and the solution was gently warmed to 30° C. (heating above 40° C. should be avoided as it increases disulfide formation). The solution was stirred in the presence of argon for 4 hours. The solution was extracted with DCM. The organic layer was dried over MgSO₄ and concentrated in vacuo. Separation by flash silica gel column chromatography afforded the title compound 16 as an orange solid (130 mg, 48%).

R_{f}=0.3, n-hexane/Dichloromethane=1:1 (v/v %).

¹H-NMR (400 MHz, CDCl₃): δ 6.07 (s, 2H), 2.98 (t, J=7.9 Hz, 2H), 2.59 (q, J=7.2 Hz, 2H), 2.53 (s, 6H), 2.44 (s, 6H), 1.66 (m, 6H), 1.36 (t, J=7.8 Hz, 1H); LRMS (ES-API): [M+H]⁺ calcd for C₁₈H₂₆BF₂N₂S 351.2 found 351.1.

### Example 3.2. Synthesis of Fluorescent Dye and Tetrazine-Introduced Nanoprobe (Compound 22)

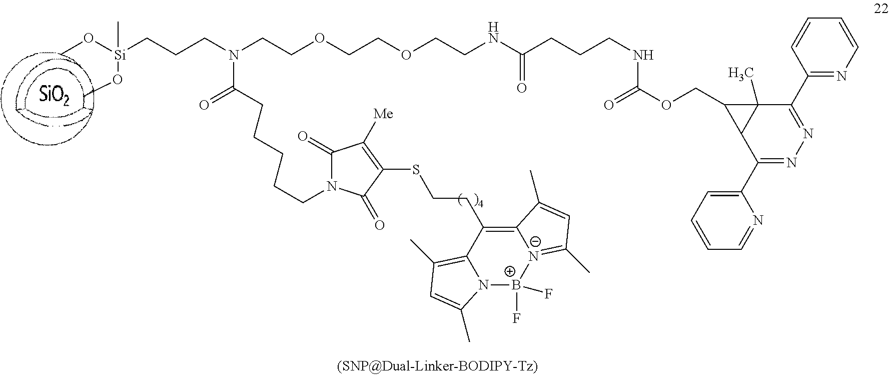

BODIPY-thiol (compound 16, 1 mg, 3.0 μmol) and Et₃N (2 μL, 1.0 μmol) were added to silica nanoparticles@Dual-Linker (compound 20, 10 mg) in CH₃CN (4 mL). The mixture was stirred at room temperature for 30 minutes. Thereafter, tetrazine (compound 18, 0.7 mg, 3.0 μmol) was added and the mixture was stirred at room temperature for 3 hours. The mixture was centrifuged. Thereafter, the supernatant was discarded and the remaining material was dispersed in CH₃CN. Sonication was performed for 10 minutes. The above described process was repeated three times (until unreacted compounds 16 or 18 were not detected by TLC).

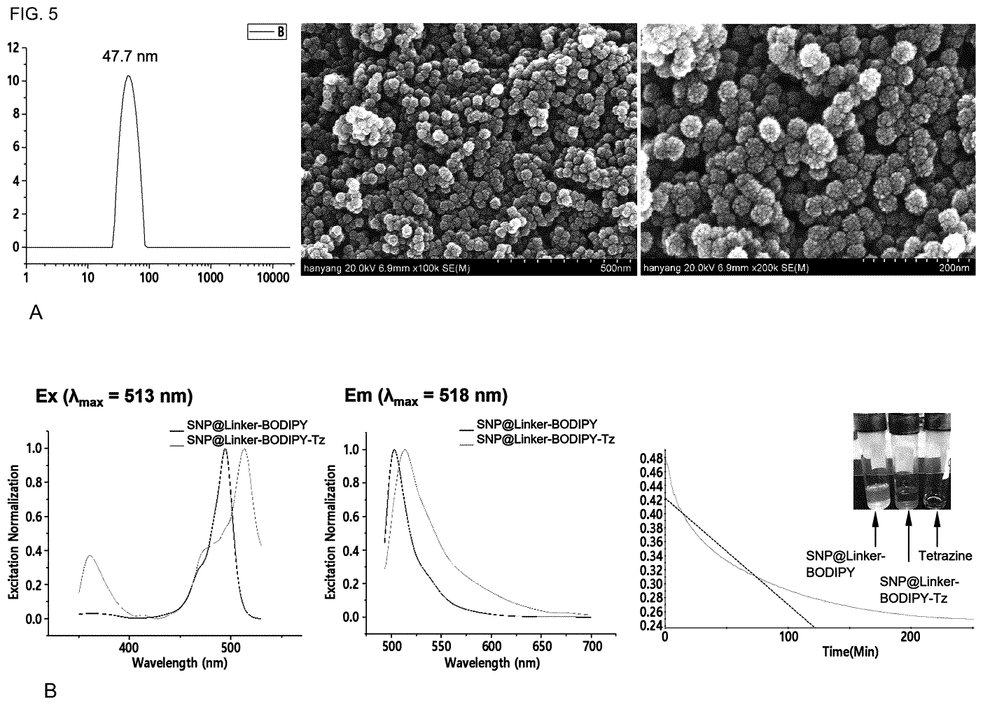

Size and shape were characterized using dynamic light scattering (DLS) and SEM (FIG. 5a). The results showed that the size of the nanoprobes (47.7 nm) remained consistent without particle aggregation.

### Example 4. Preparation of Nanoprobe of the Invention

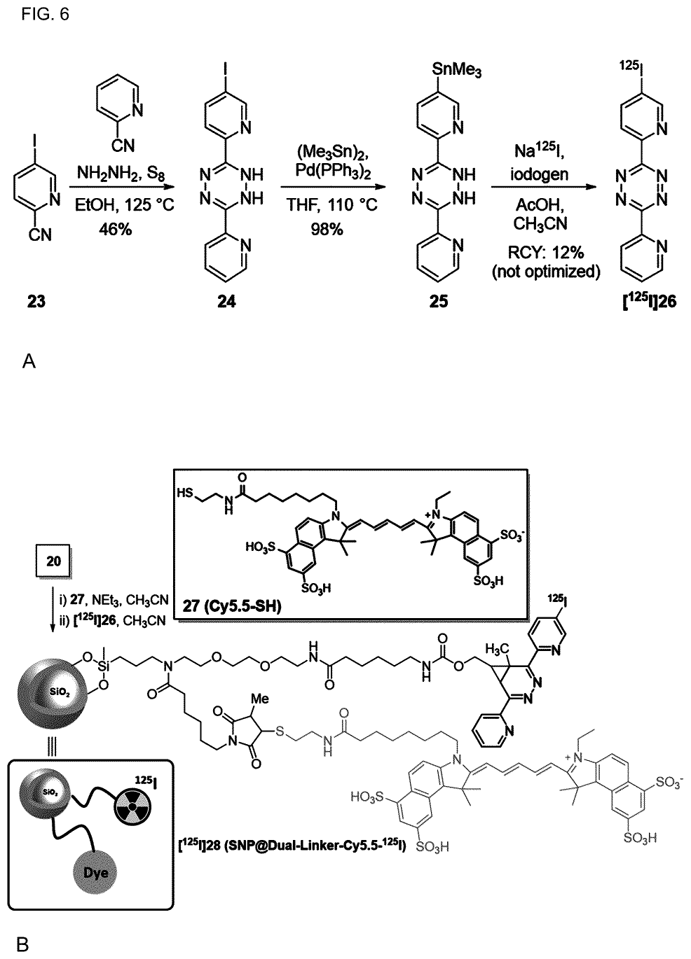

A method of producing a nanoprobe into which a fluorescent dye and a radioactive isotope are introduced according to an embodiment of the present disclosure is shown in FIG. 6.

### Example 4.1. Synthesis of Radiolabeled Tetrazine (Compound 26)

### 4.1.1. Preparation of 5-iodopicolinonitrile (Compound 23)

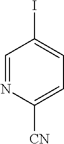

p-toluenesulfonic acid monohydrate (958 mg, 5.04 mmol) was dissolved in acetonitrile (6.7 mL), and then 5-amino-pyridine-2-carbonitrile (200 mg, 1.68 mmol) was added. The temperature was lowered to 5° C., and sodium nitrite (232 mg, 3.36 mmol), potassium iodide (724 mg, 4.37 mmol) and distilled water (1.5 mL) were added. The mixture was stirred for 10 minutes. Then, the mixture was stirred at room temperature for 4 hours. After the reaction (monitored by TLC) reached completion, an aqueous sodium bicarbonate solution was added until the pH value reached 9-10, and then a saturated sodium thiosulfate solution was added. The mixture was extracted using ethyl acetate (3×10 mL) and washed with water and saline. The organic layer was dried over anhydrous MgSO₄, and then the solvent was removed under reduced pressure. The mixture was purified by silica gel column chromatography (hexane/EtOAc=4:1) to obtain the title compound (Compound 23, 234 mg, 62%) as a pure orange solid.

R_{f}=0.55, hexane/EtOAc=4:1

¹H-NMR (400 MHz, CDCl₃) δ 8.95 (d, J=1.4 Hz, 1H), 8.19 (dd, J=8.1, 2.1 Hz, 1H), 7.46 (dd, J=8.1, 0.6 Hz, 1H)

### 4.1.2. Preparation of 3-(5-iodopyridin-2-yl)-6-(pyridin-2-yl)-1,2-dihydro-1,2,4,5-tetrazine (Compound 24)

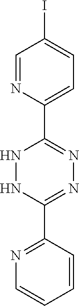 [SMILES: Ic1ccc(C2=NN=C(c3ccccn3)NN2)nc1]

Picolinonitrile (190 mg, 1.83 mmol), 5-iodopicolinonitrile (Compound 23, 100 mg, 0.43 mmol) and sulfur (28 mg, 0.87 mmol) were added to a round bottom flask filled with argon. Hydrazine hydrate 50-64% (447 μL, 7.17 mmol) and ethanol (1.7 mL) were added. The mixture was stirred at 80° C. for 2 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (hexane/EtOAc=4:1) to obtain the title compound (Compound 24, 73 mg, 46%) as a pure orange solid.

¹H-NMR (400 MHz, CDCl₃) δ 8.79 (s, 1H), 8.61 (s, 1H), 8.57 (d, J=4.6 Hz, 1H), 8.40 (s, 1H), 8.06 (t, J=7.6 Hz, 2H), 7.83 (d, J=8.4 Hz, 1H), 7.77 (t, J=7.7 Hz, 1H), 7.36 (t, J=6.1 Hz, 1H); ¹³C-NMR (100 MHz, CDCl₃) 154.6, 148.4, 147.4, 146.6, 146.5, 146.2, 145.2, 137.0, 125.1, 122.9, 121.5, 95.0.

### 4.1.3. Preparation of 3-(pyridin-2-yl)-6-(5-(trimethylstannyl)pyridin-2-yl)-1,2-dihydro-1,2,4,5-tetrazine (Compound 25)

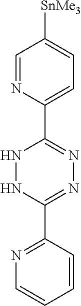

In a round bottom flask filled with argon, 1,1,1,2,2,2-hexamethyldistannane (90 mg, 0.27 mmol) was dissolved in THE (3 mL), then Pd(PPh₃)₄ (3.2 mg, 0.0027 mmol) and 3-(5-iodopyridin-2-yl)-6-(pyridin-2-yl)-1,2-dihydro-1,2,4,5-tetrazine (Compound 24, 50 mg, 0.14 mmol) were added. The mixture was stirred at 110° C. for 4 hours. After the reaction (monitored by TLC) reached completion, distilled water (10 mL) was added to terminate the reaction. The mixture was extracted using dichloromethane (3×15 mL) and washed with water and brine. The organic layer was dried over anhydrous MgSO₄, and then the solvent was removed under reduced pressure. The mixture was purified by silica gel column chromatography (hexane/EtOAc=4:1) to obtain the title compound (Compound 25, 54 mg, 98%) as a pure orange solid.

R_{f}=0.33, hexane/EtOAc=4:1

¹H-NMR (400 MHz, CDCl₃) δ 8.69 (s, 1H), 8.64 (s, 1H), 8.56 (m, 2H), 8.04 (d, J=7.9 Hz, 1H), 7.97 (d, J=7.6 Hz, 1H), 7.84 (d, J=7.6 Hz, 1H), 7.75 (td, J=7.4, 0.3 Hz, 1H), 7.35 (td, J=5.8, 0.2 Hz, 1H).

### 4.1.4. Preparation of 3-(5-(iodo-¹²⁵I)pyridin-2-yl)-6-(pyridin-2-yl)-1,2,4,5-tetrazine ([¹²⁵I] Compound 26)

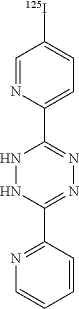 [SMILES: [125I]c1ccc(C2=NN=C(c3ccccn3)NN2)nc1]

A 5 mg/mL solution (29.3 μL) containing iodogen in 5% acetic acid/acetonitrile was added to a vial containing stanyl tetrazine (compound 25, 58.7 μL in 10 mg/mL solution in acetonitrile). The mixture was combined with aqueous sodium iodide [I-125](41 μL, 74 MBq) obtained in a 0.1 M NaOH solution. The mixture was stirred by hand for a few seconds and left for 5 minutes. To this mixture, an additional aliquot (164.5 μL) of the iodogen solution was added and the mixture was left for an additional 15 minutes. The reaction mixture was quenched with aqueous sodium thiosulfate (100 μL, 0.1 M) and extracted with dichloromethane (100 μL). The organic solvent was separated and evaporated at room temperature to obtain the title compound ([¹²⁵I] Compound 26).

### Example 4.2. Synthesis of Fluorescent Dye and Radioactive Isotope-Introduced Nanoprobe (Compound 28)

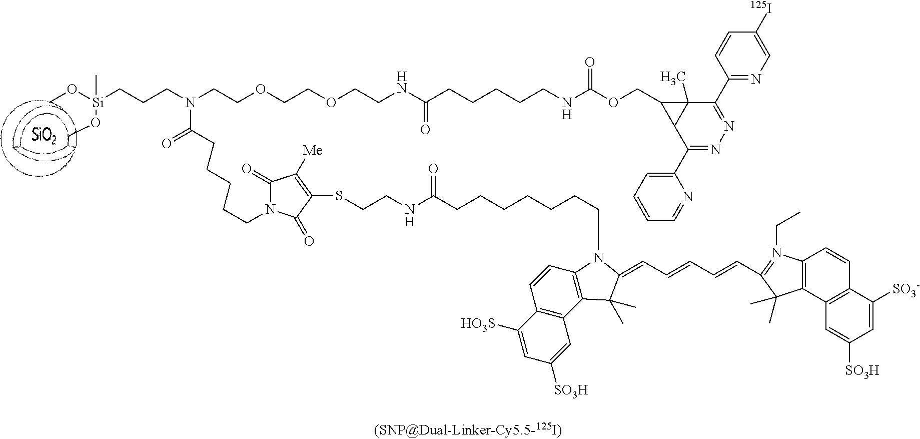

Cy5.5-thiol (compound 27, 1 mg, 0.001 mmol) and triethylamine (1 μL, 0.0005 mmol) were added to the silica nanoparticles@Dual-Linker (compound 20, 5 mg/2 mL) dispersed in acetonitrile. The mixture was stirred at room temperature for 30 minutes. Then, 3-(5-(iodo-¹²5I)pyridin-2-yl)-6-(pyridin-2-yl)-1,2,4,5-tetrazine (Compound 26, 0.7 mg, 0.003 mmol) was added. The mixture was sonicated for 1 hour at room temperature. The reaction mixture was transferred to a 1 mL plastic vial. Centrifugation was performed. Thereafter, the supernatant was discarded and a fresh acetonitrile (1 mL each) was added. Ultrasonication was performed at maximum intensity for 10 minutes. The above described process was repeated three times. TLC was used to confirm whether there was any remaining BODIPY-thiol or tetrazine. Dynamic light scattering (DLS) was used to check for the aggregation of particles.

### Experimental Example 1. Confirmation of Dual-Molecular Imaging Effect of Nanoprobes of the Invention

A Cy5.5 and ¹²⁵I-introduced nanoprobes (Compound 28) according to one embodiment of the present disclosure was administered intravenously to C57BL/6 mice, and then in vivo biodistribution imaging was sequentially performed.

All protocols were approved by the Institutional Animal Care and Use Committee of Seoul National University Bundang Hospital (IACUC No. BA-2009-304-085-04), and the Jiseokyoung Research Center is accredited by the AAALAC. All animals were maintained in accordance with the 8th edition of the ILAR Guide for the Care and Use of Laboratory Animals.

SPECT/CT images and fluorescence images were acquired immediately, 24 hours and 48 hours after intravenous injection of the nanoprobe of the present disclosure (compound 28, 100 μCi) through the tail vein of C57BL/6 mice. The activity of silica nanoparticles was measured with a dose calibrator before and after the injection. An animal SPECT/CT scanner (NanoSPECT/CT; Bioscan Inc., Washington DC, USA) was used to acquire SPECT/CT images. High-resolution static scans of the mouse head were acquired in helical scan mode with 24 projections after 60 min using a 4-head scanner with a 4×9 (1.4 mm) pinhole collimator. The energy window was set to 140 keV±15%. Subsequently, CT was used to acquire SPECT images at the same location. IVIS Lumina III (PerkinElmer, Waltham, Massachusetts, USA) was used to acquire fluorescence images. The analysis software PMOD (version 4.2, Technologies, Zurich, Switzerland) was used for image analysis. The organ volume of interest (VOI) was calculated using CT images, and the activity of each organ was measured using the VOI of each organ.

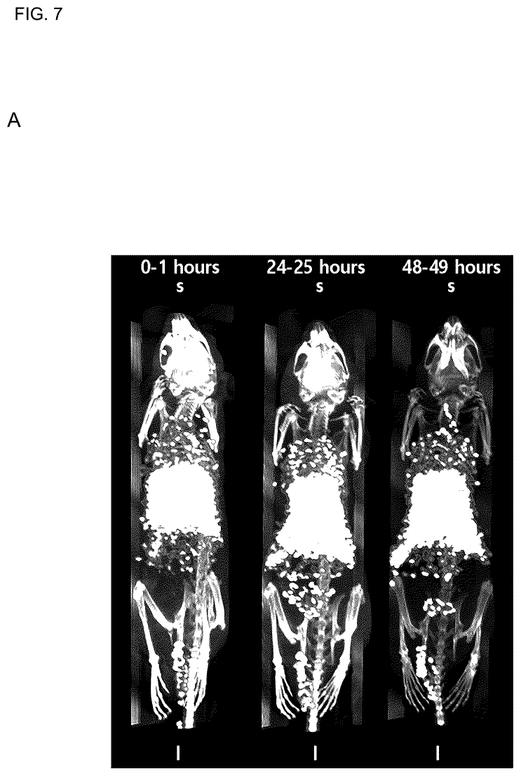

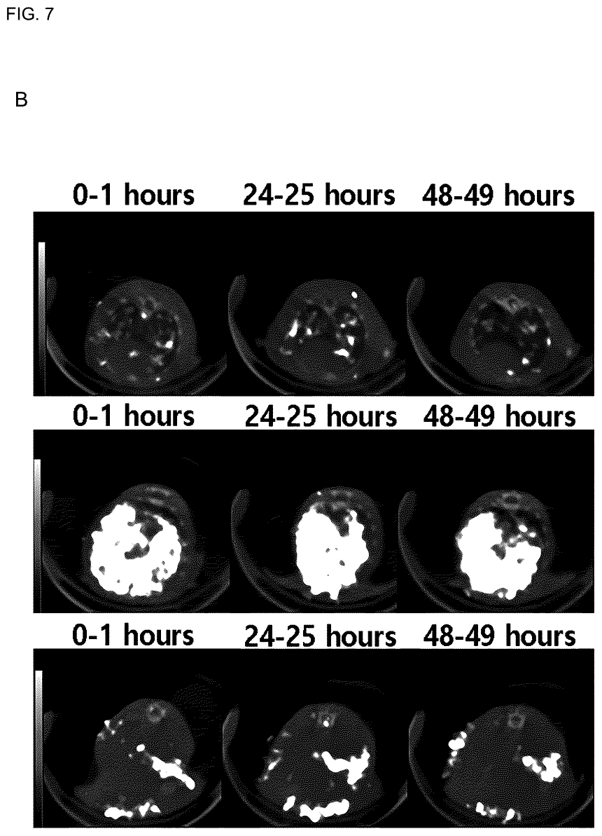

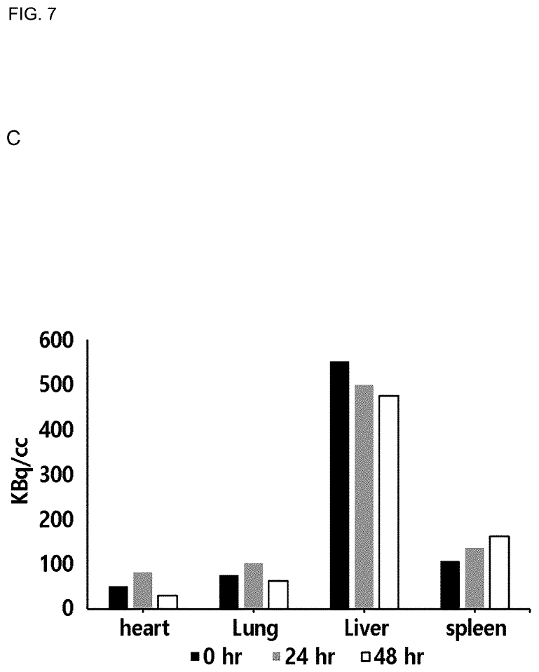

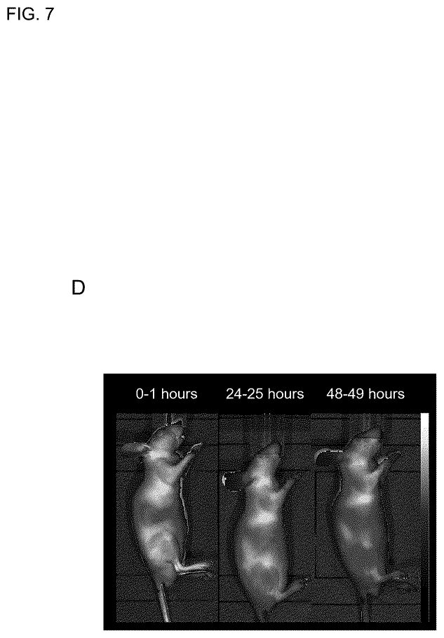

After intravenous administration, the nanoprobes (Compound 28) of the present disclosure mostly accumulated in the liver, the amount of nanoprobes in the liver gradually decreased over time, the amount of nanoprobes in the liver had decreased after 24 hours and had decreased after 48 hours, the amount of nanoprobes in the lungs and heart had increased after 24 hours and had decreased after 48 hours, and the amount of nanoprobes in the spleen had increased after 48 hours (FIGS. 7a and 7b). In terms of the stability of the nanoprobes, the total radioactivity detected in PET/CT images did not change significantly over a 48 hour period (FIG. 7c). In the thyroid gland, no absorption of ¹²⁵I was observed at all, and these results indicate that after 48 hours the developed nanoprobes had barely degraded. In the optical imaging experiments, most of the nanoprobes were detected in the liver, and overall, the biodistribution based on fluorescence imaging was comparable to the SPECT/CT imaging results (FIG. 7d). This suggests that the dual imaging nanoprobes of the present disclosure may circulate in vivo for a long period of time without being degraded.

While the embodiments are described, it will be apparent to one of ordinary skill in the art that various alterations and modifications in form and details may be made in these embodiments without departing from the spirit and scope of the claims and their equivalents. For example, suitable results may be achieved if the described techniques are performed in a different order, and/or if components in a described system, architecture, device, or circuit are combined in a different manner, or replaced or supplemented by other components or their equivalents.

Therefore, other implementations, other embodiments, and equivalents to the claims are also within the scope of the following claims.

### INDUSTRIAL APPLICABILITY

The present disclosure relates to a methallylsilane-based linker compound, a method for preparing same, and the like. The present disclosure makes it possible to prepare a nanoprobe in which two or more functional molecules are incorporated in a silica nanoparticle that is modified with the linker compound by a one-pot process through a continuous bioorthogonal reaction. The nanoprobe of the present disclosure may incorporate a targeting ligand capable of delivering a drug to a specific location, a fluorescent dye for molecular imaging, a radioactive isotope, a magnetic substance, a drug for treatment, and the like, and thus may be used in the field of diagnosis and treatment of various diseases, such as in vivo molecular imaging, theranostics, and the like.

1. A linker compound represented by the following formula: 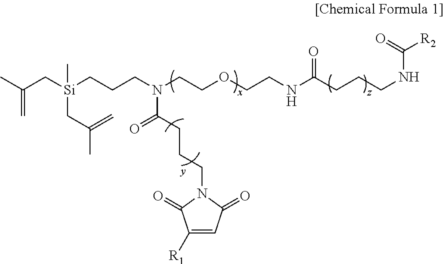
wherein,
x, y, and z are the same or different from each other, and are each independently an integer from 1 to 10,
R₁ is a chained alkyl group of C₁-C₆,
R₂ is an —OR₃ group, —O(CH₂)R₄ group, or —NH(CH₂)ₙC(O)R₅ group,
(wherein n is an integer from 1 to 5),
R₃, R₄, and R₅ are the same or different from each other, and are each independently a cycloalkene group, a cycloalkyne group, a heterocycloalkene group, or a heterocycloalkyne group of C₃-C₂₀ (wherein the cycloalkene group, cycloalkyne group, heterocycloalkene group, or heterocycloalkyne group is capable of being unsubstituted or substituted with a chained alkyl group of C₁-C₆).

2. The linker compound of claim 1, wherein each of x, y, and z in Chemical Formula 1 is 2, and
R₂ is at least one selected from the group consisting of 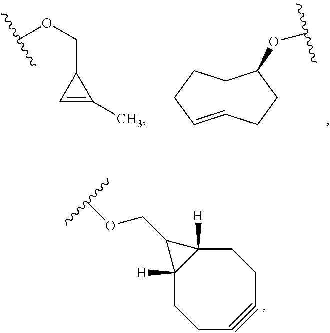
and a combination thereof.

3. The linker compound of claim 1, wherein the linker compound is a bifunctional linker.

4. (canceled)

5. A silica nanoparticle having a surface that is modified with the linker compound of claim 1.

6. (canceled)

7. A nanoprobe comprising:
the silica nanoparticles of claim 5; and
two or more functional molecules.

8. The nanoprobe of claim 7, wherein the functional molecules are one or more selected from the group consisting of a fluorescent dye, a radioactive isotope, a magnetic substance, a ligand, a drug, a polyethylene glycol (PEG), and a combination thereof.

9. The nanoprobe of claim 8, wherein the fluorescent dye is one or more selected from the group consisting of 6-carboxyfluorescein (FAM), digoxigenin (DIG), fluorescein isothiocyanate (FITC), texas red, fluorescein, 2′,4′,5′,7′-tetrachloro-6-carboxy-4,7-dichlorofluorescein (HEX), fluorescein chlorotriazinyl, rhodamine green, rhodamine red, tetramethyl rhodamine, oregon green, alexa fluor, 6-Carboxy-4′,5′-Dichloro-2′,7′-Dimethoxyfluorescein (JOE), 6-Carboxyl-XRhodamine (ROX), Tetrachloro-Fluorescein (TET), tertramethylrodamine isothiocyanate (TRITC), 6-carboxytetramethyl-rhodamine (TAMRA), N-(1-Naphthyl) ethylenediamine (NED), thiadicarbocyanine, cyanine-based dye, BODIPY-based dye, coumarin-based dye, methylene blue, and a combination thereof.

10. The nanoprobe of claim 8, wherein the radioactive isotope is one or more selected from the group consisting of ¹⁸F, ¹¹C, ¹³C, ¹³N, ¹⁵O, ⁶⁰Cu, ⁶⁴Cu, ⁶⁷Cu, ¹²⁴I, ⁶⁸Ga ⁵²Fe, ⁵⁸Co, ³H, ¹⁴C, ³⁵S, ³²P, ¹³¹, ⁵⁹Fe, ⁶⁰Co, ⁸⁹Sr, ⁹⁰Sr, ⁹⁰Y ⁹⁹Mo, ¹³³Xe, ¹³⁷Cs, ¹⁵³Sm, ¹⁷⁷Lu, ¹⁸⁶Re, ¹²³I, ¹²⁵I, ²⁰¹Tl, ⁶⁷Ga, and a combination thereof.

11. The nanoprobe of claim 8, wherein the drug is one or more anticancer agents selected from the group consisting of paclitaxel, docetaxel, doxorubicin, sorafenib, Vemurafenib, irinotecan, cisplatin, alpharadin, mitoxantrone, cyclophosphamide, vinblastine, carboplatin, actinomycin-D, etoposide, teniposide, bisantrene, homoharringtonine, Gleevec (STI-571), 5-fluorouracil, busulfan, chlorambucil, melphalan, nitrogen mustard, nitrosourea, and a combination thereof.

12. (canceled)

13. (canceled)

14. (canceled)

15. (canceled)
